(19) United States
(12) Patent Application Publication
(10) Pub. No.: US 2020/0261748 A1
(43) Pub. Date: Aug. 20, 2020

(54) PHOTOPROTECTIVE COMPOSITIONS COMPRISING AN AQUEOUS PHASE AND A LOW MELTING POINT APOLAR WAX

(71) Applicant: L'OREAL, Paris (FR)

(72) Inventors: Claude WILLEMIN, Paris (FR); Vanessa DUFFET, Joinville-le-Pont (FR)

(73) Assignee: L'OREAL, Paris (FR)

(21) Appl. No.: 16/865,882

(22) Filed: May 4, 2020

Related U.S. Application Data
(62) Division of application No. 11/482,761, filed on Jul. 10, 2006.
(60) Provisional application No. 60/702,317, filed on Jul. 26, 2005.

(30) Foreign Application Priority Data
Jul. 11, 2005 (FR) 05/52137

(51) Int. Cl. A61Q 17/04; A61K 8/31; A61K 8/81

(52) U.S. Cl. CPC A61Q 17/04; A61K 8/8111; A61K 8/31

(57) ABSTRACT

Photoprotective cosmetic compositions having at least reduced shine, stickiness, whitening and/or fluffiness and/or enhanced SPF contain an effective amount of at least one UV radiation filtering agent, at least one aqueous phase and at least one apolar wax having a melting point greater than or equal to 30° C. and a fusion enthalpy of less than 250 J/g, formulated into a topically applicable, cosmetically acceptable carrier therefor.

### CROSS-REFERENCE TO PRIORITY/PROVISIONAL APPLICATIONS

This application is a divisional of U.S. patent application Ser. No. 11/482,761, filed Jul. 10, 2006, which claims priority under 35 U.S.C. § 119 of FR 05/52137, filed Jul. 11, 2005, and of U.S. Provisional Application No. 60/702,317, filed Jul. 26, 2005, each hereby expressly incorporated by reference and each assigned to the assignee hereof.

### BACKGROUND OF THE INVENTION

### Technical Field of the Invention

The present invention relates to photoprotective compositions comprising, formulated into a cosmetically acceptable carrier, at least one UV radiation filtering agent, at least one aqueous phase, and, characteristically, at least one apolar wax having a melting point greater than or equal to 30° C. and a fusion enthalpy below 250 J/g.

### Description of Background and/or Related and/or Prior Art

It is well known that light of wavelength from 280 nm to 400 nm can cause tanning of the human epidermis and that radiation of wavelength from 280 to 320 nm, known by the designation UV-B, causes erythemas and skin burns which may adversely affect the development of natural bronzing; this UV-B radiation must therefore be filtered.

It is also known that UV-A radiation, of wavelength from 320 to 400 nm, which causes tanning of the skin, is likely to damage the latter, notably in the case of sensitive skin or skin that is constantly exposed to sunlight. In particular, UV-A radiation causes a loss of elasticity of the skin and the appearance of wrinkles leading to premature aging. It promotes initiation of the reaction of erythema or amplifies this reaction in certain individuals and may even be the cause of phototoxic or photo-allergic reactions. It is therefore also desirable to filter the UV-A radiation.

It is therefore necessary to filter the UV-A and UV-B radiation, and cosmetic compositions for protecting the human epidermis, containing UV-A and UV-B filters, are currently available.

A great many cosmetic compositions intended for photoprotection of the skin have been proposed. They contain organic UV filters and filters of mineral origin which function, depending on their chemical nature and their physical properties, by absorption, reflection or diffusion of UV radiation. Very often they are combinations of fat-soluble and/or water-soluble organic filters together with nanopigments of metal oxides such as TiO₂ or ZnO.

Sunscreen compositions are quite often in the form of an emulsion of the oil-in-water type (i.e., a cosmetically and/or dermatologically acceptable carrier comprising an aqueous continuous dispersing phase and a discontinuous dispersed oil phase) or water-in-oil (aqueous phase dispersed in a continuous oil phase), which contains, at various concentrations, one or more conventional lipophilic organic filters and/or conventional hydrophilic organic filters capable of selectively absorbing the harmful UV rays, these filters (and the amounts thereof) being selected as a function of the required sun protection factor, the sun protection factor (SPF) being expressed mathematically by the ratio of the dose of UV radiation required to reach the erythemogenic threshold with the UV filter, to the dose of UV radiation required to reach the erythemogenic threshold without a UV filter. In such emulsions, the hydrophilic filters are contained in the aqueous phase and the lipophilic filters are contained in the oil phase. Moreover, oil-in-water emulsions have better sensory qualities than the water-in-oil emulsions, with a less oily feel on application to the skin.

One of the main drawbacks of these compositions is the shiny appearance that they impart to the skin after application. This appearance is connected with the presence of filters and with the nature of the agents for dissolving them.

To combat this undesirable shiny appearance, currently known matting agents are generally used, namely inorganic fillers (such as SiO₂, ZnO), coated or uncoated, starch particles (Dry-Flo Plus from National Starch), beads of polyamide-6 or 12 (Orgasol R) or beads of high melting point polyethylene. To be effective, these matting agents must be added in high proportions. At these high concentrations they tend to give rise to downy and whitening effects on the skin.

Furthermore, the use of certain matting agents such as polyethylene waxes renders the manufacture of sunscreen emulsions containing same more restrictive and expensive, as these high melting point waxes must be used at temperatures far higher than the emulsification temperature.

Another drawback that must be taken into account is the difficulty of achieving a very high sun protection index. For this, it proves beneficial to add a high concentration of organic and/or inorganic filters. However, increasing the proportion of the organic filters has the disadvantage of leaving a shiny film on the skin that is cosmetically unacceptable and is disliked by the users, and may give rise to intolerance effects of the skin. Moreover, increasing the proportion of mineral filters such as nanopigments of a metal oxide (TiO₂) may produce an unsightly white residual film on the skin. Most often this effect is connected with poor dispersion of the nanopigments in the formulation and a heterogeneous distribution on the skin.

To increase the protection indices, film-forming polymers are generally used, but most often they cause impairment of the cosmetic properties of the formulation because it leaves a sticky, greasy and sometimes shiny film on the skin or it becomes fluffy on application.

Another possibility is to add mineral particles, but they too present the drawback that they also lead to formulations that become fluffy on application.

Yet another possibility is to add waxes such as the C₁₈-C₃₈ alkylhydroxystearyl stearate waxes described in EP-924,447, glyceride waxes indicated in EP-1-000,611, but these make the formulations shiny on application.

Thus, need continues to exist for compositions and in particular sunscreen emulsions that can be manufactured under mild conditions, which are non-shiny on application, do not cause whitening, are non-sticky, do not become fluffy, and provide excellent filtering efficiency.

### SUMMARY OF THE INVENTION

It has now unexpectedly and surprisingly been found that it is possible to ameliorate or avoid the various drawbacks described above by formulating at least one apolar wax into a carrier comprising at least one aqueous phase and a UV radiation filtering system. In addition, sunscreen compositions containing such a combination also provide good sunscreen effectiveness, and good resistance to water, sweat and washing, as well as being long-lasting.

This discovery forms the basis of the present invention.

Thus, the present invention features compositions comprising, formulated into a cosmetically acceptable carrier, at least one UV radiation filtering agent, at least one aqueous phase, and at least one apolar wax having a melting point above 30° C. and a fusion enthalpy below 250 J/g.

This invention also features formulating at least one apolar wax having a melting point above 30° C. and a fusion enthalpy below 250 J/g into compositions comprising, in a cosmetically acceptable carrier, at least one aqueous phase, with the aim of reducing or even eliminating the shine and/or reducing or eliminating the stickiness and/or reducing or eliminating the whitening and/or reducing or eliminating the fluffiness and/or increasing the sun protection factor (SPF) of such compositions.

Other characteristics, aspects and advantages of the invention will become clear from the detailed description that follows.

By “UV radiation filtering agent” is meant any single organic or inorganic compound that filters UV radiation or a mixture of several organic or inorganic compounds that filter UV radiation, for example a mixture comprising a UV-A filter and a UV-B filter.

“Cosmetically acceptable” means compatible with the skin and/or its appendages, having a pleasant color, odor and feel, and not causing unacceptable discomfort (tingling, tightness, redness) such as might deter the consumer from using this composition.

“Apolar wax” means any wax constituted only of molecules that only contain carbon and hydrogen atoms.

### DETAILED DESCRIPTION OF BEST MODE AND SPECIFIC/PREFERRED EMBODIMENTS OF THE INVENTION

The apolar waxes according to the invention are generally fat-soluble compounds, solid at room temperature (25° C.), with reversible solid/liquid change of state.

They have a melting point greater than or equal to 30° C. which can preferably be up to 80° C. and a fusion enthalpy below 250 J/g. On heating the wax to the liquid state (fusion), it is possible to make it miscible with oils and form a microscopically homogeneous mixture.

In particular, the waxes according to the invention have a melting point above 40° C. and most preferably less than or equal to 70° C.

More preferably, their fusion enthalpy is less than or equal to 240 J/g and most preferably less than or equal to 220 J/g.

The melting point of the wax and its fusion enthalpy can be measured using a differential scanning calorimeter (DSC) as marketed for example under the designation MSDSC 2929 by TA Instruments. The melting point of the compound is the temperature value corresponding to the peak of the melting curve while the fusion enthalpy corresponds to the integral of the complete melting curve, with the melting curve being measured from the start temperature and the end temperature of fusion of said wax.

The following waxes are not regarded as apolar waxes in the sense of the present invention:

(i) waxes obtained by esterification or modified by esterification and which can contain residual OH groups as a function of the esterification yield. Such waxes result for example from the reaction of a fatty acid on a branched polyol of the ditrimethylol type such as those marketed under the name HEST by HETERENE;

(ii) waxes obtained from catalytic hydrogenation of animal or vegetable oils having linear or branched C₈-C₃₂ aliphatic chains such as jojoba oil, hydrogenated sunflower oil, hydrogenated coconut oil or the wax obtained by hydrogenation of olive oil esterified with stearyl alcohol; siliconized modified waxes such as siliconized Candellila wax marketed by KOSTER KEUNEN under the name Siliconyl candellila;

(iii) hydrocarbon waxes such as beeswax, lanolin wax, orange wax, lemon wax, rice bran wax, Carnauba wax, Candellila wax, Ouricury wax, Japan wax, Berry wax, shellac wax and sumach wax; Montan wax, hydrogenated castor oil;

(iv) waxes resulting from the action of fatty acids on carbohydrates such as disaccharides of the sucrose type, such as sucrose polybehenate marketed by CRODA under the name CROMADERM B;

(v) hydroxyester waxes, for example the C₂₀-C₄₀ alkyl (hydroxystearyloxy)stearate wax or SYNTHETIC BEESWAX such as those marketed under the names “KESTER WAX K82 PR” and “KESTER WAX K80 PR”;

(vi) silicone waxes such as dimethiconol behenate and dimethiconol stearate marketed by RHODIA under the names MIRASIL WAX B and MIRASIL WAX S respectively;

(vii) apolar polyolefin waxes having a fusion enthalpy greater than 250 J/g such as the polyethylene wax marketed under the name PERFORMALENE 400 by NEW PHASE TECHNOLOGY.

Among the apolar waxes according to the invention, exemplary are the polyolefin waxes resulting from the polymerization and notably homopolymerization of α-olefin corresponding to the general formula R—CH₂═CH₂ in which R is an alkyl radical, preferably a linear alkyl radical, having from 10 to 50 carbon atoms and preferably from 25 to 50 carbon atoms.

By homopolymerization of α-olefin, we mean the polymerization of monomers essentially comprising an α-olefin or a mixture of α-olefins. These waxes preferably have a number-average molecular weight in the range from 400 to 3000 daltons and especially from 1800 to 2700 daltons.

Such polyolefin waxes are described in U.S. Pat. Nos. 4,060,569 and 4,239,546. These waxes are notably marketed under the names “PERFORMA V® 103”, “PERFORMA V® 253” AND “PERFORMA V® 260” by NEW PHASE TECHNOLOGY.

Among the apolar waxes according to the invention, also exemplary are paraffin waxes having a number-average molecular weight ranging from 350 to 600 daltons, e.g., the commercial product marketed under the name CERAFINE 56-58 (melting point: 56° C. and fusion enthalpy=232 J/g) by BAERLOCHER.

The apolar waxes according to the invention are selected more particularly from among the polymethylene waxes that can be obtained by the Fischer-Tropsch process.

They generally have a number-average molecular weight in the range from 350 to 600 daltons. The CIREBELLE waxes manufactured by SASOL can be used in particular, such as:

CIREBELLE 303: melting point 51° C. and fusion enthalpy=157-180 J/g.
CIREBELLE 305: melting point 55° C. and fusion enthalpy=185 J/g.
CIREBELLE 505: melting point 41° C. and fusion enthalpy=177 J/g.

The apolar waxes according to the invention are preferably added in a proportion from 0.1 to 5 wt. % and advantageously from 0.5 to 3.0 wt. % and 0.5 and 1.5 wt. % relative to the total weight of the composition.

The compositions according to the invention contain one or more UV radiation filtering agents selected from among the organic and/or inorganic filters that are active in the UV-A and/or the UV-B, hydrophilic and/or lipophilic and/or even insoluble in the commonly used cosmetic solvents.

The hydrophilic, lipophilic or insoluble organic UV filters are notably selected from among anthranilates; dibenzoylmethane derivatives; cinnamic derivatives; salicylic derivatives, camphor derivatives; benzophenone derivatives; derivatives of β,β-diphenylacrylate; triazine derivatives; benzotriazole derivatives; benzalmalonate derivatives notably those indicated in U.S. Pat. No. 5,624,663; benimidazole derivatives; imidazolines; bis-benzoazolyl derivatives such as are described in EP-669,323 and U.S. Pat. No. 2,463,264; derivatives of p-aminobenzoic acid (PABA); derivatives of methylene bis-(hydroxyphenyl benzotriazole) such as are described in U.S. Pat. Nos. 5,237,071, 5,166,355, GB2303549, DE-19726184 and EP-893,119; benzoxazole derivatives such as are described in EP-0-832,642, EP-1-027,883, EP-1-300,137 and DE10162844; polymer filters and silicone filters such as those described notably in WO-93/04665; dimers derived from a-alkylstyrene such as those described in DE19855649; the 4,4-diarylbutadienes such as are described in EP-0-967,200, DE19746654, DE19755649, EP-A-1-008,586, EP-1-133,980 and EP-133,981 and mixtures thereof.

Exemplary organic UV filters include those designated below under their INCI names:

Derivatives of Para-Aminobenzoic Acid:

### PABA,

### Ethyl PABA,

Ethyl dihydroxypropyl PABA,
Ethylhexyl dimethyl PABA marketed notably under the name ESCALOL 507 by ISP,

### Glyceryl PABA,

PEG-25 PABA marketed under the name UVINUL P25 by BASF.

Dibenzoylmethane Derivatives:

Butyl methoxydibenzoylmethane marketed under the trademark PARSOL 1789 by HOFFMANN LA ROCHE,
Isopropyl dibenzoylmethane.

Salicylic Derivatives:

Homosalate marketed under the name Eusolex HMS by RONA/EM INDUSTRIES,
Ethylhexyl salicylate marketed under the name NEO HELIOPAN OS by HAARMANN et REIMER,
Dipropyleneglycol salicylate marketed under the name DIPSAL by Scher, TEA salicylate, marketed under the name NEO HELIOPAN TS by HAARMANN et REIMER.

Cinnamic Derivatives:

Ethylhexyl methoxycinnamate marketed notably under the trademark PARSOL MCX by HOFFMANN LA ROCHE,
Isopropyl methoxycinnamate,
Isoamyl methoxycinnamate marketed under the trademark NEO HELIOPAN E 1000 by HAARMANN et REIMER,

### Cinoxate,

DEA methoxycinnamate,
Diisopropyl methoxycinnamate
Glyceryl ethylhexanoate methoxycinnamate.

Derivatives of β,β-Diphenylacrylate:

Octocrylene marketed notably under the trademark UVINUL N539 by BASF,
Etocrylene, marketed notably under the trademark UVINUL N35 by BASF.

Benzophenone Derivatives:

Benzophenone-1 marketed under the trademark UVINUL 400 by BASF,
Benzophenone-2 marketed under the trademark UVINUL D50 by BASF,
Benzophenone-3 or oxybenzone, marketed under the trademark UVINUL M40 by BASF,
Benzophenone-4 marketed under the trademark UVINUL MS40 by BASF,

### Benzophenone-5

Benzophenone-6 marketed under the trademark HELISORB 11 by NORQUAY
Benzophenone-8 marketed under the trademark SPECTRA-SORB UV-24 by American Cyanamid
Benzophenone-9 marketed under the trademark UVINUL DS-49 by BASF, Benzophenone-12
n-Hexyl 2-(4-diethylamino-2-hydroxybenzoyl)-benzoate marketed under the trademark UVINUL A+ by BASF.

Derivatives of Benzylidene Camphor:

3-Benzylidene camphor manufactured under the name MEXORYL SD by CHIMEX,
4-Methyl benzylidene camphor marketed under the name ESUOLEX 6300 by Merck,
Benzylidene camphor sulfonic acid manufactured under the name MEXORYL SL by CHIMEX,
Camphor benzalkonium methosulfate manufactured under the name MEXORYL SO by CHIMEX,
Terephthalylidene dicamphor sulfonic acid manufactured under the name MEXORYL SX by CHIMEX,
Polyacrylamidomethyl benzylidene camphor manufactured under the name MEXORYL SW by CHIMEX.

Derivatives of Phenyl Benzimidazole:

Phenylbenzimidazole sulfonic acid marketed notably under the trademark EUSOLEX 232 by Merck,
Disodium phenyl dibenzimidazole tetrasulfonate marketed under the trademark NEO HELIOPAN AP by HAARMANN et REIMER.

Derivatives of Phenyl Benzotriazole:

Drometrizole trisiloxane marketed under the name Silatrizole by RHODIA CHIMIE,
Methylene bis-benzotriazolyl tetramethylbutylphenol, marketed in solid form under the trademark MIXXIM BB/100 by FAIRMOUNT CHEMICAL or in micronized form in aqueous dispersion under the trademark TINOSORB M by CIBA SPECIALTY CHEMICALS.

Triazine Derivatives:

Bis-ethylhexyloxyphenol methoxyphenyl triazine marketed under the trademark TINOSORB S by CIBA GEIGY,
Ethylhexyl triazone marketed notably under the trademark UVINUL T150 by BASF,
Diethylhexyl butamido triazone marketed under the trademark UVASORB HEB by SIGMA 3V,
2,4,6-Tris-(diisobutyl 4′-amino benzalmalonate)-s-triazine,
2,4,6-Tris-(dineopentyl 4′-amino benzalmalonate)-s-triazine
the symmetrical derivatives of triazine such as those described notably in
WO2004/085412, WO2006/034991 in particular 2,4,6-tris(biphenyl)-1,3,5-triazine and 2,4,6-tris(terphenyl)-1,3,5-triazine.

Anthranilic Derivatives:

Menthyl anthranilate marketed under the trademark NEO HELIOPAN MA by HAARMANN et REIMER.

Derivatives of Imidazolines:

Ethylhexyl dimethoxybenzylidene dioxoimidazoline propionate.

Derivatives of Benzalmalonate:

Di-neopentyl-4′-methoxybenzalmalonate
Polyorganosiloxane with benzalmalonate functions such as Polysilicone-15 marketed under the trademark PARSOL SLX by HOFFMANN LA ROCHE.

Derivatives of 4,4-Diarylbutadiene:

1,1-dicarboxy-(2,2′-dimethylpropyl)-4,4-diphenylbutadiene.

Benzoxazole Derivatives:

2,4-bis-[5-1(dimethylpropyl)benzoxazol-2-yl-(4-phenyl)-imino]-6-(2-ethylhexyl)-imino-1,3,5-triazine marketed under the name Uvasorb K2A by Sigma 3V
and mixtures thereof.

The preferred organic UV radiation filtering agents are selected from among:

Ethylhexyl methoxycinnamate

Homosalate

Ethylhexyl salicylate,

Butyl methoxydibenzoylmethane

Octocrylene,

Phenylbenzimidazole sulfonic acid,

Benzophenone-3,

Benzophenone-4,

Benzophenone-5,

n-Hexyl 2-(4-diethylamino-2-hydroxybenzoyl)-benzoate.

4-Methyl benzylidene camphor,

Terephthalylidene dicamphor sulfonic acid,

Camphor Benzalkonium methosulfate,

Disodium phenyl dibenzimidazole tetrasulfonate,

Ethylhexyl triazone,

Bis-Ethylhexyloxyphenol methoxyphenyl triazine,

Diethylhexyl butamido triazone,

2,4,6-Tris-(dineopentyl 4′-amino benzalmalonate)-s-triazine

2,4,6-Tris-(diisobutyl 4′-amino benzalmalonate)-s-triazine.

2,4,6-Tris-(biphenyl)-1,3,5-triazine

2,4,6-Tris-(terphenyl)-1,3,5-triazine

Methylene bis-benzotriazolyl tetramethylbutylphenol,

Drometrizole trisiloxane

Polysilicone-15

Di-neopentyl-4′-methoxybenzalmalonate

1,1-dicarboxy-(2,2′-dimethylpropyl)-4,4-diphenylbutadiene

2,4-bis-[5-1(dimethylpropyl)benzoxazol-2-yl-(4-phenyl)-imino]-6-(2-ethylhexyl)-imino-1,3,5-triazine

and mixtures thereof.

The inorganic filters are selected from among pigments or from nanopigments (average size of the primary particles: generally from 5 nm to 100 nm, preferably from 10 nm to 50 nm) of metal oxides, coated or uncoated, for example nanopigments of oxide of titanium (amorphous or crystalline in the rutile and/or anatase form), of iron, of zinc, of zirconium or of cerium which are all well-known UV photoprotective agents.

The pigments can be coated or uncoated.

The coated pigments are pigments that have undergone one or more surface treatments of a chemical, electronic, mechanical-chemical and/or mechanical nature with compounds such as are described for example in Cosmetics & Toiletries, February 1990, Vol. 105, p. 53-64, such as amino acids, beeswax, fatty acids, fatty alcohols, anionic surfactants, lecithins, sodium, potassium, zinc, iron or aluminum salts of fatty acids, metal alkoxides (or titanium or of aluminum), polyethylene, silicones, proteins (collagen, elastin), alkanolamines, silicon oxides, metal oxides or sodium hexametaphosphate.

In known manner, the silicones are organosilicon polymers or oligomers with linear or cyclic structure, branched or crosslinked, of variable molecular weight, obtained by polymerization and/or polycondensation of appropriately functionalized silanes, and essentially comprising a repetition of main units in which the silicon atoms are joined together by oxygen atoms (siloxane bond), hydrocarbon radicals optionally substituted being directly bound by means of a carbon atom to said silicon atoms.

The term “silicones” also encompasses the silanes required for their preparation, in particular the alkyl silanes.

The silicones used for coating the nanopigments according to the present invention are preferably selected from the group comprising alkyl silanes, polydialkylsiloxanes, and polyalkylhydrogensiloxanes. More preferably, the silicones are selected from the group comprising octyl trimethyl silane, polydimethylsiloxanes and polymethylhydrogensiloxanes.

Of course, prior to their treatment with silicones, the metal oxide pigments may have been treated with other surface agents, in particular with cerium oxide, alumina, silica, aluminum compounds, silicon compounds, or mixtures thereof.

The coated pigments are more particularly titanium oxides coated:
with silica such as the product SUNVEIL from the company IKEDA,
with silica and iron oxide such as the product SUNVEIL F from the company IKEDA,
with silica and alumina such as the products MICROTITANIUM DIOXIDE MT 500 SA and MICROTITANIUM DIOXIDE MT 100 SA from the company TAYCA, TIOVEIL from the company Tioxide, and MIRASUN Ti W 60 from the company Rhodia,
with alumina such as the products TIPAQUE TTO-55 (B) and TIPAQUE TTO-55 (A) from the company ISHIHARA, and UVT 14/4 from the company KEMIRA,
with alumina and aluminum stearate such as the product MICROTITANIUM DIOXIDE MT 100 T, MT 100 TX, MT 100 Z, MT-01 from the company TAYCA, the products Solaveil CT-10 W and Solaveil CT 100 from the company UNIQEMA and the product EUSOLEX T-AVO from the company MERCK,
with silica, alumina and alginic acid such as the product MT-100 AQ from the company TAYCA,
with alumina and aluminum laurate such as the product MICROTITANIUM DIOXIDE MT 100 S from the company TAYCA,
with iron oxide and iron stearate such as the product MICROTITANIUM DIOXIDE MT 100 F from the company TAYCA,
with zinc oxide and zinc stearate such as the product BR351 from the company TAYCA,
with silica and alumina and treated with a silicone such as the products MICROTITANIUM DIOXIDE MT 600 SAS, MICROTITANIUM DIOXIDE MT 500 SAS or MICROTITANIUM DIOXIDE MT 100 SAS from the company TAYCA,
with silica, alumina, aluminum stearate and treated with a silicone such as the product STT-30-DS from the company TITAN KOGYO,
with silica and treated with a silicone such as the product UV-TITAN X 195 from the company KEMIRA,
with alumina and treated with a silicone such as the products TIPAQUE TTO-55 (S) from the company ISHIHARA, or UV TITAN M 262 from the company KEMIRA,
with triethanolamine such as the product STT-65-S from the company TITAN KOGYO,
with stearic acid such as the product TIPAQUE TTO-55 (C) from the company ISHIHARA,
with sodium hexametaphosphate such as the product MICROTITANIUM DIOXIDE MT 150 W from the company TAYCA.

Other titanium dioxide pigments treated with a silicone are preferably TiO₂ treated with octyltrimethylsilane with average size of the primary particles from 25 and 40 nm such as that marketed under the trademark T 805 by DEGUSSA SILICES, TiO₂ treated with a polydimethylsiloxane and with average size of the primary particles 21 nm, such as that marketed under the trademark 70250 CARDRE UF TIO2S13 by CARDRE, anatase/rutile TiO₂ treated with a polydimethylhydrogensiloxane and with average size of the primary particles 25 nm such as that marketed under the trademark MICRO TITANIUM DIOXYDE USP GRADE HYDROPHOBIC by COLOR TECHNIQUES.

The uncoated titanium dioxide pigments are for example marketed by TAYCA under the trademarks MICROTITANIUM DIOXIDE MT 500 B or MICROTITANIUM DIOXIDE MT 600 B, by DEGUSSA under the name P 25, by WACKHER under the name “Transparent titanium dioxide PW”, by MIYOSHI KASEI under the name UFTR, by TOMEN under the name ITS and by Tioxide under the name TIOVEIL AQ.

The uncoated zinc oxide pigments are for example
those marketed under the name Z-COTE by SUNSMART;
those marketed under the name NANOX by Elementis;
those marketed under the name NANOGARD WCD 2025 by Nanophase Technologies;

The coated zinc oxide pigments are for example
those marketed under the name “OXIDE ZINC CS-5” by TOSHIBI (ZnO coated with polymethylhydrogensiloxane);
those marketed under the name NANOGARD ZINC OXIDE FN by Nanophase Technologies (as 40% dispersion in Finsolv TN, benzoate of C₁₂-C₁₅ alcohols);
those marketed under the name DAITOPERSION Zn-30 and DAITOPERSION Zn-50 by Daito (dispersions in cyclopolymethylsiloxane/oxyethylenated polydimethylsiloxane, containing 30% or 50% of nano-oxides of zinc coated with silica and polymethylhydrogensiloxane);
those marketed under the name NFD Ultrafine ZnO by DAIKIN (ZnO coated with perfluoroalkyl phosphate and perfluoroalkylethyl-based copolymer in dispersion in cyclopentasiloxane);
those marketed under the name SPD-Z1 by SHIN-ETSU (ZnO coated with silicone-grafted acrylic polymer, dispersed in cyclodimethylsiloxane);
those marketed under the name ESCALOL Z100 by ISP (ZnO treated with alumina and dispersed in mixture of ethylhexyl methoxycinnamate/PVP-hexadecene copolymer/methicone);
those marketed under the name Fuji ZnO-SMS-10 by Fuji Pigment (ZnO coated with silica and polymethylsilsesquioxane);
those marketed under the name Nanox Gel TN by Elementis (55% dispersion of ZnO in benzoate of C₁₂-C₁₅ alcohols with polycondensate of hydroxystearic acid).

The uncoated cerium oxide pigments are marketed for example under the name COLLOIDAL CERIUM OXIDE by RHODIA. The uncoated iron oxide nanopigments are marketed for example by ARNAUD under the names NANOGARD WCD 2002 (FE 45B), NANOGARD IRON FE 45 BL AQ, NANOGARD FE 45R AQ, NANOGARD WCD 2006 (FE 45R), or by MITSUBISHI under the name TY-220.

The coated iron oxide pigments are marketed for example by ARNAUD under the names NANOGARD WCD 2008 (FE 45B FN), NANOGARD WCD 2009 (FE 45B 556), NANOGARD FE 45 BL 345, NANOGARD FE 45 BL, or by BASF under the name “TRANSPARENT IRON OXIDE”.

Also exemplary are mixtures of metal oxides, notably of titanium dioxide and cerium dioxide, including the equal-weight mixture of titanium dioxide and cerium dioxide coated with silica, marketed by IKEDA under the name SUNVEIL A, as well as the mixture of titanium dioxide and zinc dioxide coated with alumina, silica and silicone such as the product M 261 marketed by KEMIRA or coated with alumina, silica and glycerol such as the product M 211 marketed by KEMIRA.

The UV radiation filtering agents are generally present in the compositions according to the invention in proportions ranging from 0.01 wt. % to 20 wt. % relative to the total weight of the composition, and preferably ranging from 0.1 wt. % to 10 wt. % relative to the total weight of the composition.

The compositions according to the invention can also contain artificial skin bronzing and/or tanning agents (self-bronzing agents), and more particularly dihydroxyacetone (DHA). They are preferably contained in amounts ranging from 0.1 wt. % to 10 wt. % relative to the total weight of the composition.

The aqueous compositions according to the present invention can in addition contain conventional cosmetic additives notably selected from among the fats, organic solvents, ionic or non-ionic, hydrophilic or lipophilic thickeners, softeners, moisturizers, opacifiers, stabilizers, emollients, silicones, anti-foaming agents, perfumes, preservatives, anionic, cationic, non-ionic, zwitterionic or amphoteric surfactants, active ingredients, fillers, polymers, propellants, alkalizing or acidifying agents or any other ingredient usually employed in the cosmetic or dermatological field.

The fats can comprise an oil or a wax other than the apolar waxes as defined previously or their mixtures. By “oil” we mean a compound that is liquid at room temperature. By “wax” we mean a compound that is solid or substantially solid at room temperature, generally with a melting point above 35° C.

As oils, exemplary are mineral oils (paraffin); vegetable oils (sweet-almond oil, macadamia oil, blackcurrant seed oil, jojoba oil); synthetic oils such as perhydrosqualene, alcohols, aliphatic amides (such as isopropyl lauroyl sarcosinate marketed under the name ELDEW SL-205 by AJINOMOTO), fatty acids or esters (such as benzoate of C₁₂-C₁₅ alcohols marketed under the trademark FINSOLV TN or WITCONOL TN by WITCO, octyl palmitate, isopropyl lanolate, triglycerides including those of capric/caprylic acids, dicaprylyl carbonate marketed under the name Cetiol CC by COGNIS), oxyethylenated or oxypropylenated fatty esters and ethers; siliconized oils (cyclomethicone, polydimethylsiloxanes or PDMS) or fluorinated oils, polyalkylenes.

As waxy compounds, exemplary are carnauba wax, beeswax, and hydrogenated castor oil.

Among the organic solvents, exemplary are the alcohols and lower polyols. The latter can be selected from among the glycols and glycol ethers such as ethylene glycol, propylene glycol, butylene glycol, dipropylene glycol or diethylene glycol.

As hydrophilic thickeners, exemplary are the carboxyvinyl polymers such as the Carbopols (Carbomers) and the Pemulens (acrylate/C₁₀-C₃₀-alkylacrylate copolymer); polyacrylam ides such as for example the crosslinked copolymers marketed under the names SEPIGEL 305 (CTFA name: polyacrylamide/C13-14 isoparaffin/Laureth 7) or SIMULGEL 600 (CTFA name: acrylamide/sodium acryloyldimethyltaurate copolymer/isohexadecane/polysorbate 80) by SEPPIC; the polymers and copolymers of 2-acrylamido-2-methylpropane sulfonic acid, optionally crosslinked and/or neutralized, such as the poly(2-acrylamido-2-methylpropane sulfonic acid) marketed by HOECHST under the trademark HOSTACERIN AMPS (CTFA name: ammonium polyacryldimethyltauramide); the cellulose derivatives such as hydroxyethylcellulose; the polysaccharides and notably the gums such as xanthan gum; and mixtures thereof.

As lipophilic thickeners, exemplary are the synthetic polymers such as the poly C₁₀-C₃₀ alkylacrylate marketed under the name DORESCO IPA 13-1 by LANDEC or the modified clays such as hectorite and its derivatives, such as the products marketed under the name Bentone.

Among the active ingredients, exemplary are:
vitamins (A, C, E, K, PP etc.) and their derivatives or precursors, alone or as mixtures,
anti-contamination agents and/or anti-radical agent;
depigmenting agents and/or pro-pigmenting agents;
anti-glycation agents;
calmatives;
inhibitors of NO-synthase;
agents that stimulate the synthesis of dermal or epidermal macromolecules and/or prevent their degradation;
agents that stimulate the proliferation of fibroblasts;
agents that stimulate the proliferation of keratinocytes;
muscle relaxants;
tension agents;
matting agents;
keratolytic agents;
desquamating agents;
moisturizing agents;
anti-inflammatory agents;
agents acting on the energy metabolism of the cells,
insect repellants
antagonists of P substances or of CRGP.
hair restoring agents or agents against hair loss
anti-wrinkle agents.

Of course, one skilled in the art will take care in selecting the optional additional compound or compounds mentioned above and/or their amounts in such a way that the advantageous properties associated intrinsically with the compositions according to the invention will not be affected, or will not be substantially affected, by the addition or additions envisaged.

The compositions according to the invention can be formulated in accordance with techniques that are familiar to one skilled in the art. They can in particular be in the form of an emulsion, simple or complex (O/W, W/O, O/W/O OR W/O/W) such as a cream, a milk or a gel-cream; in the form of an aqueous gel; in the form of a lotion. They can optionally be packaged as an aerosol and can be supplied in the form of foam or spray.

Preferably, the compositions according to the invention are provided in the form of an oil-in-water or water-in-oil emulsion or as a dispersion.

The emulsions generally contain at least one emulsifier selected from among the amphoteric, anionic, cationic or non-ionic emulsifiers, used alone or as a mixture. The emulsifiers are selected appropriately, depending on the emulsion to be obtained (W/O or O/W).

As emulsifying surfactants that can be used for the preparation of W/O emulsions, exemplary are the alkyl esters or ethers of sorbitan, of glycerol or of sugars; the siliconized surfactants such as the dimethicone copolyols such as the mixture of cyclomethicone and dimethicone copolyol, marketed under the name DC 5225 C by DOW CORNING, and the alkyl-dimethicone copolyols such as Laurylmethicone copolyol marketed under the name DOW CORNING 5200 Formulation Aid by DOW CORNING; the cetyl dimethicone copolyol such as the product marketed under the name Abil EM 90R by GOLDSCHMIDT and the mixture of cetyl dimethicone copolyol, polyglycerol isostearate (4 mol) and hexyl laurate marketed under the name ABIL WE O9 by GOLDSCHMIDT. One or more co-emulsifiers can also be added, which can, advantageously, be selected from the group comprising the alkylated polyol esters.

As alkylated polyol esters, notably exemplary are the esters of polyethylene glycol such as PEG-30 Dipolyhydroxystearate such as the product marketed under the name ARLACEL P135 by ICI.

As esters of glycerol and/or of sorbitan, exemplary are polyglycerol isostearate, such as the product marketed under the name Isolan GI 34 by Goldschmidt; sorbitan isostearate, such as the product marketed under the name Arlacel 987 by ICI; sorbitan isostearate and glycerol, such as the product marketed under the name Arlacel 986 by ICI, and mixtures thereof.

For O/W emulsions, exemplary emulsifiers are the non-ionic emulsifiers such as the oxyalkylenated esters of fatty acids and of glycerol (more especially polyoxyethylenated); the oxyalkylenated esters of fatty acids and of sorbitan; the oxyalkylenated esters of fatty acids (oxyethylenated and/or oxypropylenated) such as the mixture PEG-100 Stearate/Glyceryl Stearate marketed for example by ICI under the name ARLACEL 165; the oxyalkylenated (oxyethylenated and/or oxypropylenated) ethers of fatty alcohols; the esters of sugars such as sucrose stearate; the ethers of fatty alcohol and of sugar, notably the alkylpolyglycosides (APG) such as decylglycoside and laurylglycoside marketed for example by Henkel under the respective names PLANTAREN 2000 and PLANTAREN 1200, ketostearylglycoside optionally mixed with ketostearyl alcohol, marketed for example under the name MONTANOV 68 by SEPPIC, under the name TEGOCARE CG90 by Goldschmidt and under the name EMULGADE KE3302 by Henkel, as well as arachidyl glycoside, for example in the form of the mixture of arachidonic and behenic alcohols and arachidylglycoside marketed under the name MONTANOV 202 by SEPPIC. According to one particular embodiment of the invention, the alkylpolyglycoside mixture as defined above with the corresponding fatty alcohol can be in the form of a self-emulsifying composition, as described for example in WO-A-92/06778.

In the case of an emulsion, the aqueous phase of the latter can comprise a non-ionic vesicular dispersion prepared according to known methods (Bangham, Standish and Watkins, J. Mol. Biol., 13, 238 (1965), FR-2-315,991 and FR-2-416,008).

The compositions according to the invention find application in a great number of treatments, whether regime or regimen, notably cosmetic, of the skin, of the lips and of the hair, including the scalp, notably for the protection and/or care of the skin, lips and/or hair, and/or for make-up of the skin and/or lips.

The present invention also features the use of the subject compositions as defined above for the manufacture of products for the cosmetic treatment of the skin, lips, nails, hair, eyelashes, eyebrows and/or of the scalp, notably care products, sunscreen products and make-up products.

The cosmetic compositions according to the invention can be used for example as care product and/or sunscreen product for the face and/or body with liquid to semi-liquid consistency, such as lotions, milks, creams that are more or less oily, gels, and gel-creams. Optionally they can be packaged as an aerosol and can be supplied in the form of foam or spray.

The cosmetic compositions according to the invention can be formulated for example as a makeup product.

The compositions according to the invention in the form of sprayable fluid lotions are applied to the skin or the hair in the form of fine particles by means of pressurizing devices.

The devices according to the invention are well known to one skilled in the art and comprise non-aerosol pumps or “atomizers”, aerosol containers comprising a propellant, as well as aerosol pumps using compressed air as propellant. The latter are described in U.S. Pat. Nos. 4,077,441 and 4,850,517.

The aerosol-packaged compositions according to the invention generally contain conventional propellants, e.g., hydrofluorinated compounds of dichlorodifluoromethane, difluoroethane, dimethylether, isobutane, n-butane, propane, trichlorofluoromethane. They are preferably contained in amounts ranging from 15 to 50 wt. % relative to the total weight of the composition.

In order to further illustrate the present invention and the advantages thereof, the following specific examples are given, it being understood that same are intended only as illustrative and in nowise limitative. In said examples to follow, all parts and percentages are given by weight, unless otherwise indicated.

The following sunscreen formulations were prepared; the amounts are stated in percentages by weight:

### Examples 1 to 3: O/W Emulsions

| | Ex 1 (*) | Ex 2 | Ex 3 (*) |
|---|---|---|---|
| Composition | wt. % | wt. % | wt. % |
| Oil phase: A | | | |
| MIXTURE OF GLYCERYL | 1 | 1 | 1 |
| MONO/DISTEARATE/ | | | |
| POLYETHYLENE GLYCOL STEARATE | | | |
| (100 Oe) | | | |
| STEARIC ACID | 1 | 1 | 1 |
| ISO-HEXADECANE | 2 | 2 | 2 |
| BENZOATE OF C12/C15 ALCOHOLS | 3 | 3 | 3 |
| CYCLOPENTASILOXANE | 5 | 5 | 5 |
| OCTOCRYLENE | 3 | 3 | 3 |
| BUTYL | 0.5 | 0.5 | 0.5 |
| METHOXYDIBENZOYLMETHANE | | | |
| TiO₂ | 1 | 1 | 1 |
| ACRYLIC ACID/STEARYL | 0.25 | 0.25 | 0.25 |
| METHACRYLATE COPOLYMER | | | |
| POLYMERIZED IN ETHYL | | | |
| ACETATE/CYCLOHEXANE MIXTURE | | | |
| XANTHAN | 0.1 | 0.1 | 0.1 |
| VINYLPYRROLIDONE/EICOSENE | 1 | 1 | 1 |
| COPOLYMER | | | |
| POLYDIMETHYLSILOXANE | 0.5 | 0.5 | 0.5 |
| VITAMIN E | 0.1 | 0.1 | 0.1 |
| POLYMETHYLENE WAX (Cirebelle | 0 | 1 | 0 |
| 303) | | | |
| SYNTHETIC BEESWAX (Kesterwax | 0 | 0 | 1 |
| K82P) | | | |
| Aqueous phase: B | | | |
| TEREPHTHALYLIDENE DICAMPHOR | 0.9 | 0.9 | 0.9 |
| SULFONIC ACID | | | |
| GLYCEROL | 6 | 6 | 6 |
| PROPYLENE GLYCOL | 6 | 6 | 6 |
| TRIETHANOLAMINE | 0.71 | 0.71 | 0.71 |
| MONOCETYL/MONO-POTASSIUM | 1 | 1 | 1 |
| PHOSPHATE | | | |
| WATER | qsf 100 | qsf 100 | qsf 100 |
| Phase C: | | | |
| PRESERVATIVE | 1.25 | 1.25 | 1.25 |
| CHELATING AGENT | 0.1 | 0.1 | 0.1 |
| PERFUME | | | 1 |

(*) not according to the invention:
Example 1 does not contain wax
Example 3 contains SYNTHETIC BEESWAX which is a polar wax of the C₂₀-C₄₀ alkyl (hydroxystearyloxy)stearate type of wax.

Method of Preparation:

Phases A and B are heated separately at 60-70° C., the emulsion is prepared with agitation of the rotor-stator type and then phase C is added. The emulsion is permitted to cool to room temperature, with moderate agitation.

Measurement of Mattness:

Reflection is measured using a gonioreflectometer on a film of thickness 30 μm previously dried for 60 minutes at room temperature.

The composition is spread on a contrast card (Prüfkarte type 24/5-250 cm² marketed by Ericksen) using a film-puller.

The result obtained is the ratio R of the specular reflection to the diffuse reflection. The value of R decreases as the matting effect increases.

| Composition | Ex 1 | Ex 2 | Ex 3 |
|---|---|---|---|
| R | 28.15 ± 1.16 | 7.93 ± 0.1 | 34.28 ± 0.1 |

At a concentration of 1%, Cirebelle 303 (composition 2) makes it possible to reduce the shine of the control formulation, whereas SYNTHETIC BEESWAX or KESTERWAX K82P increases the shine.

Filtering Efficiency:

The sun protection factor (SPF) for compositions 1, 2 and 3 was determined according to the protocol Colipa 2003.

The SPF values obtained were as follows:

| Formulation | Ex 1 | Ex 2 | Ex 3 |
|---|---|---|---|
| SPF in vivo | 5.9 ± 0.9 | 13.1 ± 2.5 | 10.7 ± 1.7 |

The results of these tests demonstrate that composition 2 according to the invention containing the apolar polymethylene wax with melting point above 51° C. and with a fusion enthalpy equal to 157-180 J/g makes it possible to reduce the shine considerably and at the same time increase the sun protection factor relative to composition 1 that does not contain wax, in contrast to composition 3 containing a polar wax.

### Example 5: W/O Sunscreen Milk

| | Ex 5 |
|---|---|
| Composition | Invention |
| Oil phase A₁ | |
| PEG-30 | 2 |
| DIPOLYHYDROXYSTEARATE | |
| C12-15 ALKYL BENZOATE | 7.5 |
| ISOHEXADECANE | 5 |
| OCTOCRYLENE | 2.5 |
| ETHYLHEXYL SALICYLATE | 5 |
| BIS-ETHYLHEXYLOXYPHENOL | 1 |
| METHOXYPHENYL TRIAZINE | |
| BUTYL | 3.5 |
| METHOXYDIBENZOYLMETHANE | |
| ETHYLHEXYL TRIAZONE | 1 |
| SYNTHETIC WAX: Cirebelle 303 | 1 |
| TiO₂ | 5 |
| Oil phase A₂ | |
| LAURYL PEG/PPG-18/18 | 1.82 |
| METHICONE | |
| ISOSTEARYL ALCOHOL | 0.18 |
| CYCLOHEXASILOXANE | 6 |
| CYCLOPENTASILOXANE | 6 |
| ETHYLHEXYLGLYCEROL | 0.5 |
| VITAMIN E | 0.2 |
| Aqueous phase | |
| TEREPHTHALYLIDENE DICAMPHOR | 1.5 |
| SULFONIC ACID | |
| TRIETHANOLAMINE | 0.78 |
| GLYCEROL | 2 |
| PROPYLENE GLYCOL | 4 |
| CHELATING AGENT | 0.3 |
| ALCOHOL | 6 |
| WATER | qsf |

Method of Preparation:

Phase A₁ (except TiO₂) is heated on a water bath at 85° C. and the aqueous phase is heated at 65° C. with stirring. The TiO₂ is dispersed in phase A₁ with rotor-stator stirring. Phase A₂ is added to A₁. The emulsion is prepared at 65-70° C. by introducing the aqueous phase with vigorous rotor-stator stirring. It is permitted to cool to room temperature before adding the alcohol.

The composition thus obtained is matt on application, it does not turn white, it is non-sticky, does not become fluffy and it gives a satisfactory filtering efficiency.

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A topically applicable photoprotective cosmetic composition comprising an effective amount of at least one UV radiation filtering agent, at least one aqueous phase and at least one apolar wax having a melting point greater than or equal to 30° C. and a fusion enthalpy of less than 250 J/g, formulated into a topically applicable, cosmetically acceptable carrier therefor.

2. The photoprotective cosmetic composition as defined by claim 1, said at least one apolar wax having a melting point ranging from 30° C. to 80° C.

3. The photoprotective cosmetic composition as defined by claim 2, said at least one apolar wax having a melting point greater than 40° C.

4. The photoprotective cosmetic composition as defined by claim 3, said at least one apolar wax having a melting point of up to 70° C.

5. The photoprotective cosmetic composition as defined by claim 1, said at least one apolar wax having a fusion enthalpy of up to 240 J/g.

6. The photoprotective cosmetic composition as defined by claim 1, said at least one apolar wax having a fusion enthalpy of up to 220 J/g.

7. The photoprotective cosmetic composition as defined by claim 1, said at least one apolar wax comprising a polyolefin wax polymerizate of an α-olefin having the formula R—CH₂═CH₂ in which R is an alkyl radical having from 10 to 50 carbon atoms.

8. The photoprotective cosmetic composition as defined by claim 7, said polyolefin wax having a number-average molecular weight ranging from 400 to 3,000 daltons

9. The photoprotective cosmetic composition as defined by claim 7, said polyolefin wax having a number-average molecular weight ranging from 1,800 to 2,700 daltons

10. The photoprotective cosmetic composition as defined by claim 1, said at least one apolar wax comprising a paraffin wax having a number-average molecular weight ranging from 350 to 600 daltons.

11. The photoprotective cosmetic composition as defined by claim 1, said at least one apolar wax comprising a polymethylene wax prepared via the Fischer-Tropsch process.

12. The photoprotective cosmetic composition as defined by claim 11, said polymethylene wax having a number-average molecular weight ranging from 350 to 600 daltons.

13. The photoprotective cosmetic composition as defined by claim 1, said at least one apolar wax comprising from 0.1% to 5% by weight thereof.

14. The photoprotective cosmetic composition as defined by claim 1, said at least one apolar wax comprising from 0.5% to 3% by weight thereof.

15. The photoprotective cosmetic composition as defined by claim 1, said at least one apolar wax comprising from 0.5% to 1.5% by weight thereof.

16. The photoprotective cosmetic composition as defined by claim 1, said at least one UV radiation filtering agent comprising an organic and/or inorganic UV filter active in the UV-A and/or UV-B range and which is hydrophilic and/or lipophilic and/or insoluble in conventional cosmetic solvents.

17. The photoprotective cosmetic composition as defined by claim 16, comprising at least one organic UV filter selected from the group consisting of anthranilates; dibenzoylmethane derivatives; cinnamic derivatives; salicylic derivatives, camphor derivatives; benzophenone derivatives; derivatives of β,β-diphenylacrylate; triazine derivatives; benzotriazole derivatives; benzalmalonate derivatives; benzimidazole derivatives; imidazolines; bis-benzoazolyl derivatives; derivatives of p-aminobenzoic acid (PABA); derivatives of methylene bis-(hydroxyphenyl benzotriazole); benzoxazole derivatives; polymer filters and silicone filters; dimers derived from α-alkylstyrene; the 4,4-diarylbutadienes and mixtures thereof.

18. The photoprotective cosmetic composition as defined by claim 17, comprising at least one organic UV filter selected from the group consisting of:
Ethylhexyl methoxycinnamate,
Homosalate,
Ethylhexyl salicylate,
Butyl methoxydibenzoylmethane,
Octocrylene,
Phenylbenzimidazole sulfonic acid,
Benzophenone-3,
Benzophenone-4,
Benzophenone-5,
n-Hexyl 2-(4-diethylamino-2-hydroxybenzoyl)-benzoate,
4-Methyl benzylidene camphor,
Terephthalylidene dicamphor sulfonic acid,
Camphor benzalkonium methosulfate,
Disodium phenyl dibenzimidazole tetrasulfonate,
Ethylhexyl triazone,
Bis-Ethylhexyloxyphenol methoxyphenyl triazine,
Diethylhexyl butamido triazone,
2,4,6-Tris-(dineopentyl 4′-amino benzalmalonate)-s-triazine,
2,4,6-Tris-(diisobutyl 4′-amino benzalmalonate)-s-triazine,
2,4,6-Tris-(biphenyl)-1,3,5-triazine,
2,4,6-Tris-(terphenyl)-1,3,5-triazine,
Methylene bis-benzotriazolyl tetramethylbutylphenol,
Drometrizole trisiloxane,
Polysilicone-15,
Di-neopentyl-4′-methoxybenzalmalonate,
1,1-Dicarboxy-(2,2′-dimethylpropyl)-4,4-diphenylbutadiene,
2,4-bis-[5-1(dimethylpropyl)benzoxazol-2-yl-(4-phenyl)-imino]-6-(2-ethylhexyl)-imino-1,3,5-triazine, and mixtures thereof.

19. The photoprotective cosmetic composition as defined by claim 16, comprising at least one inorganic UV filter selected from the group consisting of pigments and nanopigments of metal oxides, whether treated or untreated.

20. The photoprotective cosmetic composition as defined by claim 19, comprising at least one inorganic UV filter selected from the group consisting of the oxides of titanium, zinc, iron, zirconium, cerium and mixtures thereof, treated or untreated.

21. The photoprotective cosmetic composition as defined by claim 1, further comprising at least one agent for artificial bronzing and/or tanning of the skin.

22. The photoprotective cosmetic composition as defined by claim 1, further comprising at least one cosmetic additive selected from the group consisting of fats, organic solvents, ionic or non-ionic, hydrophilic or lipophilic thickeners, softeners, moisturizers, opacifiers, stabilizers, emollients, silicones, anti-foaming agents, perfumes, preservatives, anionic, cationic, non-ionic, zwitterionic or amphoteric surfactants, active ingredients, fillers, polymers, propellants, alkalizing or acidifying agents, and mixtures thereof.

23. The photoprotective cosmetic composition as defined by claim 1, formulated as an emulsion or dispersion.

24. The photoprotective cosmetic composition as defined by claim 1, formulated as a product for the cosmetic treatment of the skin, lips, nails, hair, eyelashes, eyebrows and/or scalp.

25. The photoprotective cosmetic composition as defined by claim 1, formulated as a product for the care of the skin, lips, nails, hair and/or scalp.

26. The photoprotective cosmetic composition as defined by claim 1, formulated as a makeup.

27. The photoprotective cosmetic composition as defined by claim 1, formulated as a sunscreen.

28. A regime or regimen for the UV photoprotection of the skin, lips, nails, hair, eyelashes, eyebrows and/or scalp, comprising topically applying thereon a thus effective amount of the photoprotective cosmetic composition as defined by claim 1.

29. A method for reducing or eliminating the shine and/or reducing or eliminating the stickiness and/or replacing or eliminating the whitening and/or reducing or eliminating fluffiness and/or increasing the sun protection factor (SPF) of a photoprotective cosmetic composition comprising, in a cosmetically acceptable carrier, at least one aqueous phase and at least one UV filtering agent, which comprises formulating therein at least one apolar wax having a melting point greater than or equal to 30° C. and a fusion enthalpy of less than 250 J/g.
